(19) United States
(12) Patent Application Publication
(10) Pub. No.: US 2021/0023140 A1
(43) Pub. Date: Jan. 28, 2021

(54) PLATELET FORMULATIONS AND MEDICAL USES THEREOF

(71) Applicant: PRP SCIENTIFIC, INC., San Diego, CA (US)

(72) Inventor: Jamesette Hays, II, Gardena, CA (US)

(21) Appl. No.: 16/931,193

(22) Filed: Jul. 16, 2020

Related U.S. Application Data
(60) Provisional application No. 62/879,402, filed on Jul. 26, 2019.

(51) Int. Cl. A61K 35/19; A61K 35/50; A61K 35/28; A61P 17/02; A61K 45/06; A61K 33/14; A61K 33/30; A61P 25/02; A61P 19/02; A61P 21/00

(52) U.S. Cl. CPC A61K 35/19; A61K 35/50; A61K 35/28; A61P 17/02; A61K 45/06; A61K 2035/124; A61K 33/30; A61P 25/02; A61P 19/02; A61P 21/00; A61K 33/14

(57) ABSTRACT

The present invention provides a pharmaceutical composition comprising platelets, calcium chloride, and zinc sulfate, wherein the weight ratio of calcium chloride to zinc sulfate is between about 500:1 and about 10:1. Also provided is a method of treating a wound, chronic pain, neuropathy, orthopedic injury, or musculoskeletal disease, the method comprising: administering to a subject in need thereof an effective amount of a pharmaceutical composition comprising platelets, calcium chloride, and zinc sulfate.

### CROSS-REFERENCE TO RELATED APPLICATIONS

This application claims priority to U.S. Provisional Application No. 62/879,402, filed Jul. 26, 2019, the contents of which is incorporated herein by reference in its entirety.

### TECHNICAL FIELD

This application relates to field of pharmaceutical compositions comprising activated platelets and the use of these pharmaceutical compositions to treat various diseases and injuries.

### BACKGROUND

Platelets serve a diverse role in the human body with their most important function facilitating wound healing and hemostasis. The repair response of tissue, whether dermal or musculoskeletal, generally starts with the formation of a blood clot and degranulation of platelets, which releases growth factors and cytokines at the site of injury. The wound bed passively absorbs the growth factors which then stimulate the fibroblasts within the wound bed to proliferate and secrete further cytokines as well as produce collagen.

The release of cytokines via platelets increases integrins on fibroblasts and epithelial cells, promoting their activation and proliferation. Platelets adhere to exposed matrix via integrins that bind to collagen and laminin. Kertainocytes migrate into the area and begin the restoration process of epithelium. Collagen proliferation is triggered, promoting tissue contracture and scar formation. Platelets contain a rich cytoplasm including actin, myosin, glycogen, lysosomes, and dense granules and a-granules. The secretion of a-granules include proteins like platelet-derived growth factor (PDGF), which stimulates wound healing and is a powerful mitogen for vascular smooth muscle. Another important protein secreted by platelets is von Willebrand factor, which regulates circulating levels of factor VIII.

Platelets respond rapidly to tissue damage and are triggered by ADP to aggregate at the site of the injury. A positive feedback mechanism occurs as platelets begin to secrete cytokines and various proteins which in return bind to the platelet promoting further aggregation and granule release. Platelet aggregation is also induced by platelet-activating factor (PAF), a cytokine released by platelets and other immunologic proteins. PAF stimulates the secondary messenger, G-protein, which increases the production of arachidonic acid derivates, including thromboxane Az (TXA2), which serves as a weak agonist for the activation of soluble N-ethymaleimide soluble factor receptor (SNARE). The SNARE machinery is responsible for the fusion of granule to the inner leaflet of the plasma membrane, resulting in the exocytosis of essential chemokines.

The activation of platelets via agonist can promote the release of an abundant selection of chemokines and other proteins including epidermal growth factor (EGF), adenosine diphosphate (ADP), fibronectin, fibrinogen, histamine, platelet-derived growth factor (PDGF), serotonin, and von Willebrand factor. The activation of platelets is preceded by granule release. The activation of platelets is most commonly done by introducing thrombin or calcium.

Recent therapeutic procedures have demonstrated benefits of harvesting platelets and, following their activation, injecting or applying them topically in the form of platelet-rich plasma at the site of an injured or diseased region of tissue. Platelet-rich plasma is a source of platelets which is derived from blood by sequestering and concentrating platelets by gradient density centrifugation. Platelet-rich plasma not only contains cytokines and growth factors, but has been demonstrated in the treated wound to induce the upregulation of vital growth factors including vascular endothelial growth factor (VEGF), fibroblast growth factor (FGF), EGF, PDGF and transforming growth factor beta (TGF-β). Growth factors are proteins that serve a mediatory role in the injury healing process, capable of stimulating cellular proliferation as well as cellular differentiation. Platelet-rich plasma has many applications such as accelerated healing of infected wounds, abscesses, sinuses and fistulae; osteogenesis; angiogenesis; alleviating chronic tendonitis, cartilage and disc regeneration; cardiac tissue repair; vascular disorders, venous stasis ulcers; necrosis, post-operative tissue damage; and acute and chronic injuries.

Improved methods of enhancing tissue repair and revitalization by treating a patient with a platelet-containing composition or formulation are needed. An object of the present invention is to provide a method of treating injured or diseased tissue using platelet compositions and formulations with increased efficacy. Another object of the invention is to provide a method of tissue repair and revitalization using a combination of a platelet composition or formulation and a second therapeutic composition or therapy thereby achieving a significantly improved or synergistic effect.

### SUMMARY

The present invention provides a pharmaceutical composition comprising platelets, calcium chloride, and zinc sulfate, wherein the weight ratio of calcium chloride to zinc sulfate is between about 500:1 and about 10:1, e.g., between 250:1 and 100:1. In certain aspects, the platelets are in a concentration of about 100,000/μl to about 1,000,000/μl, about 100,000/μl to about 750,000/μl, or about 100,000/μl to about 500,000/μl.

In one nonlimiting embodiment, the weight ratio of calcium chloride to zinc sulfate is between about 350:1 and about 100:1. In another embodiment, the combined concentration of calcium chloride and zinc sulfate is less than about 500 mg/mL. In yet another embodiment, the combined concentration of calcium chloride and zinc sulfate is between about 25 mg/mL and about 300 mg/mL.

In some aspects, the pharmaceutical composition further comprises cytokines, chemokines, inflammatory mediators, and growth factors. In one aspect, the pharmaceutical composition is formulated as a liquid, a solid, a semi-solid, or a combination thereof.

In other embodiments, the present invention provides a kit comprising: a collection device for collecting blood from a patient; a calcium chloride solution; a zinc sulfate solution; and instructions for centrifuging the collected blood and then adding the appropriate amount of the calcium chloride solution and of the zinc sulfate solution based on the indication to be treated in the patient.

In one aspect, the appropriate amount of the calcium chloride solution and of the zinc sulfate solution is a weight ratio of calcium chloride to zinc sulfate between about 500:1 and about 10:1.

Also disclosed is a method of treating a wound, chronic pain, neuropathy, orthopedic injury, or musculoskeletal disease, the method comprising: administering to a subject in need thereof an effective amount of a pharmaceutical composition comprising platelets, calcium chloride, and zinc sulfate, wherein the weight ratio of calcium chloride to zinc sulfate is between about 500:1 and about 10:1.

In certain embodiments, the wound is an epidermal wound, skin wound, chronic wound, acute wound, external wound, internal wound, or congenital wound. In other embodiments, the chronic pain or neuropathy is, or is caused by, neuritis, diabetes mellitus, peripheral neuropathy, reflex sympathetic dystrophy syndrome, phantom limb pain, post-amputation pain, postherpetic neuralgia, shingles, central pain syndrome, Guillain-Barre Syndrome, degenerative disc disease, cancer, multiple sclerosis, kidney disorders, alcoholism, human immunodeficiency virus-related neuropathy, Wartenberg's Migratory Sensory Neuropathy, fibromyalgia syndrome, causalgia, spinal cord injury, or exposure to a chemical agent. In yet other embodiments, the orthopedic injury is a bone defect, disc herniation or degenerative disc disease. In one embodiment, the musculoskeletal disease is selected from the group consisting of shoulder, elbow, hip, neck, or foot pain, osteoarthritis, osteoporosis, tendinitis, bursitis, gout, fibromyalgia, rheumatoid arthritis, degenerative changes to muscles, tendons, ligaments, or joints, reduced strength, sarcopenia, and soft tissue rheumatism.

In other aspects, the method further comprises administering to the subject a second therapeutic agent or a second therapy, wherein the combination of the pharmaceutical composition and the second therapeutic agent or second therapy results in a synergistic effect.

In one aspect, the second therapeutic agent or second therapy is administered simultaneously with the pharmaceutical composition. In another aspect, the second therapeutic agent or second therapy is administered sequentially with the pharmaceutical composition.

In some embodiments, the second therapeutic agent comprises stem cells. In certain embodiments, the stem cells are amniotic stem cells or adipose tissue-derived stem cells.

### BRIEF DESCRIPTION OF THE DRAWINGS

FIG. 1 depicts an injection body diagram used to guide administration of the platelet compositions and formulations.

### DETAILED DESCRIPTION

### Platelet Compositions and Formulations

The composition may comprise platelets derived from a human or animal source of whole blood. The compositions may be prepared from an autologous source, an allogenic source, a single source, or a pooled source of platelets and/or plasma. To derive the composition, whole blood may be collected, for example, using a blood collection syringe. The amount of blood collected may depend on a number of factors, including, for example, the amount of platelets desired, the health of the patient, the severity or location of the tissue disease or damage, or any suitable combination of factors.

Any suitable amount of blood may be collected. For example, about 20 cc to about 150 cc of blood may be drawn. More specifically, about 27 cc to about 110 cc or about 27 cc to about 55 cc of blood may be withdrawn. In some embodiments, the blood may be collected from a patient who may be presently suffering, or who has previously suffered from, tissue disease or damage. Platelet compositions or formulations made from a patient's own blood may significantly reduce the risk of adverse reactions or infection.

In one embodiment, about 55 cc of blood may be withdrawn into a 60 cc syringe (or another suitable syringe) that contains about 5 cc of an anticoagulant, such as a citrate dextrose solution. The syringe may be attached to an apheresis needle, and primed with the anticoagulant. Blood (about 27 cc to about 55 cc) may be drawn from the patient using standard aseptic practice. In some embodiments, a local anesthetic such as anbesol, benzocaine, lidocaine, procaine, bupivicaine, or any appropriate anesthetic known in the art may be used to anesthetize the insertion area.

The platelet compositions or formulations may be prepared in several suitable ways.

For example, the platelet compositions or formulations may be prepared from whole blood using a centrifuge. The whole blood may or may not be cooled after being collected. Isolation of platelets from whole blood depends upon the density difference between platelets and red blood cells. The platelets and white blood cells are concentrated in the layer (i.e., the “buffy coat”) between the platelet depleted plasma (top layer) and red blood cells (bottom layer). For example, a bottom buoy and a top buoy may be used to trap the platelet-rich layer between the upper and lower phase. This platelet-rich layer may then be withdrawn using a syringe or pipette.

In some embodiments, the blood may then be centrifuged using a gravitational platelet system, such as the Cell Factor Technologies GPS System® centrifuge. The blood-filled syringe containing between about 20 cc to about 150 cc of blood (e.g., about 55 cc of blood) and about 5 cc citrate dextrose may be slowly transferred to a disposable separation tube which may be loaded into a port on the GPS centrifuge. The sample may be capped and placed into the centrifuge. The centrifuge may be counterbalanced with about 60 cc sterile saline, placed into the opposite side of the centrifuge. Alternatively, if two samples are prepared, two GPS disposable tubes may be filled with equal amounts of blood and citrate dextrose. The samples may then be spun to separate platelets from blood and plasma. The samples may be spun at about 2000 rpm to about 5000 rpm for about 5 minutes to about 30 minutes. For example, centrifugation may be performed at about 3000 rpm, about 3100 rpm, about 3200 rpm, about 3300 rpm, about 3400 rpm, about 3500 rpm, about 3600 rpm, about 3700 rpm, about 3800 rpm, about 3900 rpm, or about 4000 rpm for extraction from a side of the separation tube and then isolated platelets may be suspended in about 3 cc to about 5 cc of plasma by agitation. In one embodiment, centrifugation is performed at about 3300 rpm. The platelet composition or formulation may then be extracted from a side port using, for example, a 10 cc syringe. If about 55 cc of blood may be collected from a patient, about 5 cc of platelet composition or formulation may be obtained.

In certain embodiments, calcium chloride is added to the composition or formulation as a platelet activator. In another embodiment, the composition or formulation also comprises zinc as a platelet aggregator. The zinc significantly enhances platelet aggregation and results in an increased delivery of activated platelets containing growth factors to the affected area. The combination of calcium chloride and zinc sulfate in particular weight ratios is used in the “Activation/Aggregation Solution” that forms part of the platelet compositions and formulations.

TABLE 1
Nonlimiting examples of weight ratios of zinc sulfate
and calcium chloride in Activation/Aggregation
Solution and frequency of administration according
to disease or indication to be treated.

| | Range of | |
|---|---|---|
| | Weight Ratios | |
| | (Calcium | |
| Disease or | Chloride to | |
| Indication | Zinc Sulfate) | Frequency of Administration |
| Wound | 500:1 to 10:1 | Every 72 hours; assess wound at each |
| Healing | | administration and continue until wound |
| (e.g., ulcers) | | has healed. |
| Chronic Pain | 350:1 to 100:1 | Weekly administration for the first 2-3 |
| | | weeks followed by administration once |
| | | every two weeks for at least 4-6 weeks. |
| Neuropathy | 350:1 to 100:1 | Weekly administration for the first 2-3 |
| | | weeks followed by administration once |
| | | every two weeks for at least 4-6 weeks. |
| Orthopedic | 500:1 to 10:1 | Acute injuries: weekly administration |
| Injuries | | for at least 3-4 weeks. |
| | | Chronic injuries: administration once |
| | | every two weeks for at least 6 weeks. |
| Musculo- | 500:1 to 10:1 | Administration once every two weeks |
| skeletal | | for at least 6 weeks. |
| Disease | | |

In some aspects, the weight ratio of calcium chloride to zinc sulfate is about 1000:1 to about 1:1, about 900:1 to about 1:1, about 800:1 to about 1:1, about 700:1 to about 1:1, about 600:1 to about 1:1, about 500:1 to about 1:1, about 400:1 to about 1:1, about 300:1 to about 1:1, about 200:1 to about 1:1, or about 100:1 to about 1:1. In other aspects, the weight ratio of calcium chloride to zinc sulfate is about 1000:1 to about 100:1, about 900:1 to about 100:1, about 800:1 to about 100:1, about 700:1 to about 100:1, about 600:1 to about 100:1, about 500:1 to about 100:1, about 400:1 to about 100:1, about 300:1 to about 100:1, or about 200:1 to about 100:1.

In certain embodiments, the Activation/Aggregation Solution comprises calcium chloride at a concentration of at least 5 mg/mL, at least 10 mg/mL, at least 15 mg/mL, at least 20 mg/mL, at least 25 mg/mL, at least 30 mg/mL, at least 35 mg/mL, at least 40 mg/mL, at least 45 mg/mL, at least 50 mg/mL, at least 55 mg/mL, at least 60 mg/mL, at least 65 mg/mL, at least 70 mg/mL, at least 75 mg/mL, at least 80 mg/mL, at least 85 mg/mL, at least 90 mg/mL, at least 95 mg/mL, or at least 100 mg/mL calcium chloride. In other embodiments, the Activation/Aggregation Solution comprises calcium chloride at a concentration of between about 5 mg/mL and about 250 mg/mL, between about 10 mg/mL and about 200 mg/mL, between about 25 mg/mL and about 200 mg/mL, between about 25 mg/mL and about 100 mg/mL, or between about 50 mg/mL and about 100 mg/mL.

In certain embodiments, the Activation/Aggregation Solution comprises zinc sulfate at a concentration of at least 5 mg/mL, at least 10 mg/mL, at least 15 mg/mL, at least 20 mg/mL, at least 25 mg/mL, at least 30 mg/mL, at least 35 mg/mL, at least 40 mg/mL, at least 45 mg/mL, at least 50 mg/mL, at least 55 mg/mL, at least 60 mg/mL, at least 65 mg/mL, at least 70 mg/mL, at least 75 mg/mL, at least 80 mg/mL, at least 85 mg/mL, at least 90 mg/mL, at least 95 mg/mL, or at least 100 mg/mL calcium chloride. In other embodiments, the Activation/Aggregation Solution comprises zinc sulfate at a concentration of between about 5 mg/mL and about 250 mg/mL, between about 10 mg/mL and about 200 mg/mL, between about 25 mg/mL and about 200 mg/mL, between about 25 mg/mL and about 100 mg/mL, or between about 50 mg/mL and about 100 mg/mL.

In yet other embodiments, the combined concentration of calcium chloride and zinc sulfate in the Activation/Aggregation Solution is less than about 500 mg/mL, less than about 400 mg/mL, less than about 300 mg/mL, less than about 200 mg/mL, or less than about 100 mg/mL. In one aspect, the combined concentration of calcium chloride and zinc sulfate in the Activation/Aggregation Solution is between about 25 mg/mL and about 500 mg/mL, between about 25 mg/mL and about 400 mg/mL, between about 25 mg/mL and about 300 mg/mL, between about 25 mg/mL and about 200 mg/mL, or between about 25 mg/mL and about 100 mg/mL.

In certain aspects, the Activation/Aggregation Solution is added to the collected plasma after centrifugation at a dilution of Activation/Aggregation Solution to plasma (v/v) of at least about 1:1, at least about 1:2, at least about 1:3, at least about 1:4, at least about 1:5, at least about 1:6, at least about 1:7, at least about 1:8, at least about 1:9, at least about 1:10. In some embodiments, the dilution of Activation/Aggregation Solution to plasma (v/v) after centrifugation is between about 1:1 and 1:20, between about 1:1 and 1:15, between about 1:1 and 1:10, between about 1:1 and 1:5, between about 1:5 and 1:20, between about 1:5 and 1:15, or between about 1:5 and 1:10.

As the disclosed composition or formulation comprises activated platelets, active agents within the platelets are released. These agents include, but are not limited to, cytokines (e.g., IL-1B, IL-6, TNF-A), chemokines (e.g., ENA-78 (CXCL5), IL-8 (CXCL8), MCP-3 (CCL7), MIP-1A (CCL3), NAP-2 (CXCL7), PF4 (CXCL4), RANTES (CCL5)), inflammatory mediators (e.g., PGE2), and growth factors (e.g., Angiopoitin-1, bFGF, EGF, FGF, HGF, IGF-I, IGF-II, PDAF, PDEGF, PDGF AA and BB, TGF-β1, 2, and 3, and VEGF).

The disclosed composition or formulation may be delivered as a liquid, a solid, a semi-solid (e.g., a gel), an inhalable powder, or some combination thereof. When the composition or formulation is delivered as a liquid, it may comprise a solution, an emulsion, a suspension, etc. A semi-solid or gel may be prepared by adding a clotting agent (e.g., thrombin) to the composition or formulation containing activated platelets. The gel may be more viscous than a solution and therefore may better preserve its position once it is delivered to target tissue.

In some instances, it may be desirable to deliver the composition or formulation as a liquid and have it gel or harden in situ. For example, the compositions or formulations may include, for example, collagen, cyanoacrylate, adhesives that cure upon injection into tissue, liquids that solidify or gel after injection into tissue, suture material, agar, gelatin, light-activated dental composite, other dental composites, silk-elastin polymers, MATRIGEL® (gelatinous protein mixture) (BD Biosciences), hydrogels and/or other suitable biopolymers. Alternatively, the above-mentioned agents need not form part of the composition or formulation. For example, the above-mentioned agents may be delivered to the target tissue before or after the composition or formulation has been delivered to the target tissue to cause the composition or formulation to gel. In some embodiments, the composition or formulation may harden or gel in response to one or more environmental or chemical factors such as temperature, pH, proteins, etc.

The composition or formulation may be buffered using an alkaline buffering agent to a physiological pH. The buffering agent may be a biocompatible buffer such as HEPES, TRIS, monobasic phosphate, monobasic bicarbonate, or any suitable combination thereof that may be capable of adjusting the composition or formulation to physiological pH between about 6.5 and about 8.0. In certain embodiments, the physiological pH may be from about 7.3 to about 7.5, and may be about 7.4. For example, the buffering agent may be a sodium bicarbonate solution.

As noted above, the disclosed composition or formulation may comprise one or more additional agents, diluents, solvents, or other ingredients. Examples of the additional agents include, but are not limited to, thrombin, epinephrine, collagen, calcium salts, pH adjusting agents, materials to promote degranulation or preserve platelets, additional growth factors or growth factor inhibitors, NSAIDS, steroids, anti-infective agents, and mixtures and combinations of the foregoing.

Furthermore, the disclosed compositions and formulation may comprise a contrast agent for detection by an imaging technique such as X-rays, magnetic resonance imaging (MRI), or ultrasound. Examples of such contrast agents include, but are not limited to, X-ray contrast (e.g., ISOVUE-M® (iopamidol)), MRI contrast (e.g., gadolinium), and ultrasound contrast.

### Wound Healing

In another embodiment of the methods of treatment described herein, the disclosed composition or formulation is used for the treatment of a wound, including but not limited to: an epidermal wound, skin wound, chronic wound, acute wound, external wound, internal wound, or a congenital wound (e.g., dystrophic epidermolysis bullosa).

In other embodiments, the disclosed composition or formulation is administered to a subject for the treatment of a wound infection, e.g., a wound infection followed by a breakdown of a surgical or traumatic wound. The compositions and formulations described herein have therapeutic utility in the treatment of wound infections from any microorganism known in the art, e.g., microorganisms that infect wounds originating from within the human body, which is a known reservoir for pathogenic organisms, or from environmental origin. A non-limiting example of the microorganisms, the growth of which in wounds may be reduced or prevented by the methods and compositions described herein are Staphylococcus aureus, S. epidermidis, beta hemolytic Streptococci, Escherichia coli, Klebsiella and Pseudomonas species, and among the anaerobic bacteria, Clostridium welchii and C. tartium, which are the cause of gas gangrene, mainly in deep traumatic wounds.

In other embodiments, the disclosed composition or formulation is administered for the treatment of burns, including but not limited to, first-degree burns, second-degree burns (partial thickness burns), third degree burns (full thickness burns), infection of burn wounds, infection of excised and unexcised burn wounds, infection of grafted wound, infection of donor site, loss of epithelium from a previously grafted or healed burn wound or skin graft donor site, and burn wound impetigo.

In particular, the compositions and formulations described herein have enhanced utility in the treatment of ulcers, e.g., leg ulcers. In various embodiments, said leg ulcer can be, for example, a venous leg ulcer, arterial leg ulcer, diabetic leg ulcer, decubitus ulcer, or split thickness skin grafted ulcer or wound. In this context, “treatment of a leg ulcer” comprises contacting the leg ulcer with an amount of the disclosed composition or formulation effective to improve at least one aspect of the leg ulcer. As used herein, “aspect of the leg ulcer” includes objectively measurable parameters such as ulcer size, depth or area, degree of inflammation, ingrowth of epithelial and/or mesodermal tissue, gene expression within the ulcerated tissue that is correlated with the healing process, quality and extent of scarring etc., and subjectively measurable parameters, such as patient well-being, perception of improvement, perception of lessening of pain or discomfort associated with the ulcer, patient perception that treatment is successful, and the like.

Provided herein are methods for the treatment of venous leg ulcers comprising administering an amount of the disclosed composition or formulation effective to improve at least one aspect of the venous leg ulcer. Venous leg ulcers, also known as venous stasis ulcers or venous insufficiency ulcers, a type of chronic or non-healing wound, are widely prevalent in the United States, with approximately 7 million people, usually the elderly, afflicted. Worldwide, it is estimated that 1-1.3% of individuals suffer from venous leg ulcers. Approximately 70% of all leg ulcers are venous ulcers.

Venous leg ulcers are often located in the distal third of the leg known as the gaiter region, and typically on the inside of the leg. The ulcer is usually painless unless infected. Venous leg ulcers typically occur because the valves connecting the superficial and deep veins fail to function properly. Failure of these valves causes blood to flow from the deep veins back out to the superficial veins. This inappropriate flow, together with the effects of gravity, causes swelling and progression to damage of lower leg tissues. Patients with venous leg ulcers often have a history of deep vein thrombosis, leg injury, obesity, phlebitis, prior vein surgery, and lifestyles that require prolonged standing. Other factors may contribute to the chronicity of venous leg ulcers, including poor circulation, often caused by arteriosclerosis; disorders of clotting and circulation that may or may not be related to atherosclerosis; diabetes; renal (kidney) failure; hypertension (treated or untreated); lymphedema (buildup of fluid that causes swelling in the legs or feet); inflammatory diseases such as vasculitis, lupus, scleroderma or other rheumatological conditions; medical conditions such as high cholesterol, heart disease, high blood pressure, sickle cell anemia, or bowel disorders; a history of smoking (either current or past); pressure caused by lying in one position for too long; genetics (predisposition for venous disease); malignancy (tumor or cancerous mass); infections; and certain medications.

Thus, in another embodiment, provided herein is a method of treating a venous leg ulcer comprising contacting the venous leg ulcer with an amount of the disclosed composition or formulation sufficient to improve at least one aspect of the venous leg ulcer. In another specific embodiment, the method additionally comprises treating an underlying cause of the venous leg ulcer.

The methods for treating a venous leg ulcer provided herein further encompass treating the venous leg ulcer by administering a therapeutically effective amount of the disclosed composition or formulation, in conjunction with one or more therapies or treatments used in the course of treating a venous leg ulcer. The one or more additional therapies may be used prior to, concurrent with, or after administration of the disclosed composition or formulation. In some embodiments, the one or more additional therapies comprise compression of the leg to minimize edema or swelling. In some embodiments, compression treatments include wearing therapeutic compression stockings, multilayer compression wraps, or wrapping an ACE bandage or dressing from the toes or foot to the area below the knee.

Arterial leg ulcers are caused by an insufficiency in one or more arteries' ability to deliver blood to the lower leg, most often due to atherosclerosis. Arterial ulcers are usually found on the feet, particularly the heels or toes, and the borders of the ulcer appear as though they have been ‘punched out’. Arterial ulcers are frequently painful. This pain is relieved when the legs are lowered with feet on the floor as gravity causes more blood to flow into the legs. Arterial ulcers are usually associated with cold white or bluish, shiny feet.

The treatment of arterial leg ulcers contrasts to the treatment of venous leg ulcers in that compression is contraindicated, as compression tends to exacerbate an already-poor blood supply, and debridement is limited, if indicated at all. Thus, in another embodiment, provided herein is a method of treating an arterial leg ulcer comprising treating the underlying cause of the arterial leg ulcer, e.g., arteriosclerosis, and contacting the arterial leg ulcer with an amount of the disclosed composition or formulation sufficient to improve at least one aspect of the arterial leg ulcer. In a specific embodiment, the method of treating does not comprise compression therapy.

Diabetic foot ulcers are ulcers that occur as a result of complications from diabetes. Diabetic ulcers are typically caused by the combination of small arterial blockage and nerve damage, and are most common on the foot, though they may occur in other areas affected by neuropathy and pressure. Diabetic ulcers have characteristics similar to arterial ulcers but tend to be located over pressure points such as heels, balls of the feet, tips of toes, between toes or anywhere bony prominences rub against bed sheets, socks or shoes.

Treatment of diabetic leg ulcers is generally similar to the treatment of venous leg ulcers, though generally without compression; additionally, the underlying diabetes is treated or managed. Thus, in another embodiment, provided herein is a method of treating a diabetic leg ulcer comprising treating the underlying diabetes, and contacting the diabetic leg ulcer with an amount of the disclosed composition or formulation sufficient to improve at least one aspect of the diabetic leg ulcer.

Decubitus ulcers, commonly called bedsores or pressure ulcers, can range from a very mild pink coloration of the skin, which disappears in a few hours after pressure is relieved on the area to a very deep wound extending into the bone. Decubitus ulcers occur frequently with patients subject to prolonged bedrest, e.g., quadriplegics and paraplegics who suffer skin loss due to the effects of localized pressure. The resulting pressure sores exhibit dermal erosion and loss of the epidermis and skin appendages. Factors known to be associated with the development of decubitus ulcers include advanced age, immobility, poor nutrition, and incontinence. Stage 1 decubitus ulcers exhibit nonblanchable erythema of intact skin. Stage 2 decubitus ulcers exhibit superficial or partial thickness skin loss. Stage 3 decubitus ulcers exhibit full thickness skin loss with subcutaneous damage. The ulcer extends down to underlying fascia, and presents as a deep crater. Finally, stage 4 decubitus ulcers exhibit full thickness skin loss with extensive destruction, tissue necrosis, and damage to the underlying muscle, bone, tendon or joint capsule. Thus, in another embodiment, provided herein is a method of treating a decubitus leg ulcer comprising treating the underlying diabetes, and contacting the decubitus leg ulcer with an amount of the disclosed composition or formulation sufficient to improve at least one aspect of the decubitus leg ulcer.

The methods of treatment provided herein further encompass treating a leg ulcer by administering the disclosed composition or formulation in conjunction with one or more therapies or treatments used in the course of treating a leg ulcer. The one or more additional therapies may be used prior to, concurrent with, or after administration of the disclosed composition or formulation. The disclosed composition or formulation and one or more additional therapies, may be used where the disclosed composition or formulation, alone, or the one or more additional therapies, alone, would be insufficient to measurably improve, maintain, or lessen the worsening of, one or more aspects of a leg ulcer.

In specific embodiments, the one or more additional therapies comprise, without limitation, treatment of the leg ulcer with a wound healing agent (e.g., platelet-derived growth factor (PDGF), REGRANEX® (becaplermin)); administration of an anti-inflammatory compound; administration of a pain medication; administration of an antibiotic; administration of an anti-platelet or anti-clotting medication; application of a prosthetic; application of a dressing (e.g., moist to moist dressings; hydrogels/hydrocolloids; alginate dressings; collagen-based wound dressings; antimicrobial dressings; composite dressings; synthetic skin substitutes, etc.), and the like. For any of the above embodiments, in a specific embodiment, the leg ulcer is a venous leg ulcer, a decubitus ulcer, a diabetic ulcer, or an arterial leg ulcer.

In another specific embodiment, the additional therapy is a pain medication. In another embodiment, therefore, the method of treating a leg ulcer comprises contacting the leg ulcer with the disclosed composition or formulation, and administering a pain medication to lessen or eliminate leg ulcer pain. In a specific embodiment, the pain medication is a topical pain medication.

In another specific embodiment, the additional therapy is an anti-infective agent. Preferably, the anti-infective agent is one that is not cytotoxic to healthy tissues surrounding and underlying the leg ulcer; thus, compounds such as iodine and bleach are disfavored. Thus, treatment of the leg ulcer, in one embodiment, comprises contacting the leg ulcer with the disclosed composition or formulation, and administering an anti-infective agent. The anti-infective agent may be administered by any route, e.g., topically, orally, buccally, intravenously, intramuscularly, anally, etc. In a specific example, the anti-infective agent is an antibiotic, a bacteriostatic agent, antiviral compound, a virustatic agent, antifungal compound, a fungistatic agent, or an antimicrobial compound. In another specific embodiment, the anti-infective agent is ionic silver. In a more specific embodiment, the ionic silver is contained within a hydrogel. In specific embodiments, the leg ulcer is a venous leg ulcer, arterial leg ulcer, decubitus ulcer, or diabetic ulcer.

### Chronic Pain and Neuropathy

Chronic pain, e.g., neuropathic pain, a condition that afflicts at least 30% of Americans, is caused, e.g., by disorders of the nervous system, also known as neuropathy, and can be accompanied by, or caused by, tissue damage, including nerve fibers that are damaged, dysfunction or injured. Neuropathic pain may also be caused by, e.g. pathologic lesions, neurodegeneration processes, or prolonged dysfunction of parts of the peripheral or central nervous system. However, neuropathic pain can also be present when no discernible tissue damage is evident.

Neuropathic pain is generally regarded as having two components: central plasticity, e.g., as a result of changes in receptor population or receptor sensitivity at any level of the CNS, and changes in peripheral nerves, neurons and microglial, which are mediators of central sensitization of the spinal cord. Such sensitization is known to play a major role in mediating chronic inflammatory pain and neuropathic pain.

Thus, in another aspect, provided herein is a method of treating a subject having chronic pain comprising administering to the subject a therapeutically-effective amount of a composition or formulation as described herein. In a specific embodiment, the chronic pain, e.g., neuropathic pain, is, or is caused by, neuritis (e.g., polyneuritis, brachial neuritis, optic neuritis, vestibular neuritis, cranial neuritis, or arsenic neuritis), diabetes mellitus (e.g., diabetic neuropathy), peripheral neuropathy, reflex sympathetic dystrophy syndrome, phantom limb pain, post-amputation pain, postherpetic neuralgia, shingles, central pain syndrome (pain caused, e.g., by damage to the brain, spinal cord and/or brainstem), Guillain-Barre Syndrome, degenerative disc disease, cancer, multiple sclerosis, kidney disorders, alcoholism, human immunodeficiency virus-related neuropathy, Wartenberg's Migratory Sensory Neuropathy, fibromyalgia syndrome, causalgia, spinal cord injury, or exposure to a chemical agent, e.g., trichloroethylene or dapsone (diaminyl-diphenyl sulfone).

In specific embodiments, the peripheral neuropathy is mononeuropathy (damage to a single peripheral nerve); polyneuropathy (damage to more than one peripheral nerve, frequently sited in different parts of the body), mononeuritis multiplex (simultaneous or sequential damage to noncontiguous nerve trunks), or autonomic neuropathy. Peripheral neuropathy, e.g., mononeuritis multiplex, may be caused by, e.g., diabetes mellitus, vasculitis (e.g., polyarteritis nodosa, Wegener granulomatosis, or Churg-Strauss syndrome), rheumatoid arthritis, lupus erythematosus (SLE), sarcoidosis, an amyloidosis, or cryoglobulinemia.

As used herein, “therapeutically effective amount” is an amount of the composition or formulation sufficient to result in a detectable, or reportable, lessening of said chronic pain. The lessening of pain may be, e.g., self-reported by the individual, or may be determined by physiological signs responsive to pain, e.g. elevated blood pressure, anxiety, and the like. Levels of neuropathic pain may be assessed, e.g., by the Visual Analog Scale (VAS). Numeric Pain Intensity Scale, Graphic Rating Scale, Verbal Rating Scale, Pain Faces Scale (Faces Pain Scale), Numeric Pain Intensity & Pain Distress Scales, Brief Pain Inventory (BPI), Memorial Pain Assessment, Alder Hey Triage Pain Score, Dallas Pain Questionnaire, Dolorimeter Pain Index (DPI), Face Legs Activity Cry Consolability Scale, Lequesne Scale, McGill Pain Questionnaire (MPQ), Descriptor differential scale (DDS), Neck Pain and disability Scale (NPAD), Numerical 11-Point Box (BS-11), Roland-Morris Back Pain Questionnaire, or the Wong-Baker FACES Pain Rating Scale. An improvement after administration of the composition to the subject in one or more of these assessments of pain is considered therapeutically effective.

In a specific embodiment, the disclosed composition or formulation is administered to said subject locally, e.g., at one or more sites of, or adjacent to, nerve damage that causes said chronic pain, e.g., neuropathic pain. In certain specific embodiments, the composition is administered epicutaneously, subsutaneously, intradermally, subdermally, intramuscularly, intranasally, intrathecally, intraperitoneally, intraosseously, intravesically, epidurally, intracerebrally, intracerebroventricularly, or the like. In certain specific embodiments, the composition is administered locally within 0.5, 1.0, 2.0, 3.0, 4.0 or 5.0 cm from the site of an injury that causes or is associated with neuropathic pain, or from the site of nerve injury that causes or is associated with neuropathic pain. In certain other specific embodiments, the composition is administered locally within 0.5, 1.0, 2.0, 3.0, 4.0 or 5.0 cm from the site of perceived pain, e.g., that area or areas on the individual's body in which the subject perceived the neuropathic pain.

The composition or formulation can be, for example, administered locally, distally from a site of neuropathic pain, to a nerve or set of nerves that serve a damaged area of the body of an individual, e.g., an area of the body in which the subject is experiencing the neuropathic pain. For example, the composition or formulation can be administered along the spine at any point at which nerve trunks emerge from the spinal column, e.g., any of the cervical nerves, thoracic nerves, or lumbar nerves. In specific embodiments, the composition can be administered adjacent to the spinal cord at which point nerves emerging at C1, C2, C3, C4, C5, C6, or C7, or T1, T2, T3, T4, T5, T6, T7, T8, T9, T10, T11 or T12, or L1, L2, L3, L4 or L5, or at the sacrum.

### Orthopedic Injuries and Ailments

In another specific embodiment of the methods of treatment described herein, the disclosed composition or formulation is used for the treatment of orthopedic injuries and ailments, including but not limited to, bone defects, disc herniation and degenerative disc disease.

In a particular aspect, provided herein is a method for treating a bone defect in a subject, comprising administering to a subject in need thereof a therapeutically effective amount of an implantable or injectable composition or formulation as described herein sufficient to treat the bone defect in the subject. In certain embodiments, the bone defect is an osteolytic lesion associated with a cancer, a bone fracture, or a spine, e.g., in need of fusion.

In certain embodiments, the osteolytic lesion is associated with multiple myeloma, bone cancer, or metastatic cancer. In certain embodiments, the bone fracture is a non-union fracture. In certain embodiments, an implantable composition or formulation as described herein is administered to the subject. In certain embodiments, an implantable composition or formulation as described herein is surgically implanted, e.g., at the site of the bone defect. In certain embodiments, an injectable composition or formulation as described herein is administered to the subject. In certain embodiments, an injectable composition or formulation as described herein is surgically administered to the region of the bone defect.

In particular, the disclosed composition or formulation described herein has enhanced utility in the treatment of herniated discs and degenerative disc disease. In some embodiments, the degenerative disc disease is characterized on X-ray tests or MRI scanning of the spine as a narrowing of the normal “disc space” between the adjacent vertebrae.

Disc degeneration, medically referred to as spondylosis, can occur with age when the water and protein content of the cartilage of the body changes. This change results in weaker, more fragile and thin cartilage. Because both the discs and the joints that stack the vertebrae (facet joints) are partly composed of cartilage, these areas are subject to degenerative changes, which renders the disc tissue susceptible to herniation. The gradual deterioration of the disc between the vertebrae is referred to as degenerative disc disease.

Degeneration of the disc can cause local pain in the affected area, for example, radiculopathy, i.e., nerve irritation caused by damage to the disc between the vertebrae. In particular, weakness of the outer ring leads to disc bulging and herniation. As a result, the central softer portion of the disc can rupture through the outer ring of the disc and abut the spinal cord or its nerves as they exit the bony spinal column.

Any level of the spine can be affected by disc degeneration. Thus, in some embodiments, the degenerative disc disease treatable by the methods provided herein is cervical disc disease, i.e., disc degeneration that affects the spine of the neck, often accompanied by painful burning or tingling sensations in the arms. In some embodiments, the degenerative disc disease is thoracic disc disease, i.e., disc degeneration that affects the mid-back. In some embodiments, the degenerative disc disease is lumbago, i.e., disc degeneration that affects the lumbar spine.

In particular embodiments, the method for treating degenerative disc disease in a subject comprises administering to a subject in need thereof a therapeutically effective amount of an implantable or injectable composition or formulation as described herein sufficient to treat cervical or lumbar radiculopathy in the subject. In some embodiments, the lumbar radiculopathy is accompanied by incontinence of the bladder and/or bowels. In some embodiments, the method for treating degenerative disc disease in a subject comprises administering to a subject in need thereof a therapeutically effective amount of an implantable or injectable composition or formulation as described herein sufficient to relieve sciatic pain in the subject.

In some embodiments of the methods of treating disc degeneration in a subject with a composition or formulation as provided herein, disc degeneration of the subject occurs between C1 and C2; between C2 and C3; between C3 and C4; between C4 and C5; between C5 and C6; between C6 and C7; between C7 and T1; between T1 and T2; between T2 and T3; between T3 and T4; between T4 and T5; between T5 and T6; between T6 and T7; between T7 and T8; between T8 and T9; between T9 and T10; between T10 and T11; between T11 and T12; between T12 and L1; between L1 and L2; between L2 and L3; between L3 and L4; or between L4 and L5.

Degenerative arthritis (osteoarthritis) of the facet joints is also a cause of localized lumbar pain that can be detected with plain X-ray testing. Wear of the facet cartilage and the bony changes of the adjacent joint is referred to as degenerative facet joint disease or osteoarthritis of the spine.

The methods for treating degenerative disc disease provided herein further encompass treating degenerative disc disease by administering a therapeutically effective amount of a composition or formulation as provided herein, in conjunction with one or more therapies or treatments used in the course of treating degenerative disc disease. The one or more additional therapies may be used prior to, concurrent with, or after administration of the composition or formulation as provided herein. In some embodiments, the one or more additional therapies comprise administration of medications to relieve pain and muscles spasm, cortisone injection around the spinal cord (epidural injection), physical therapy (heat, massage, ultrasound, electrical stimulation), and rest (not strict bed rest, but avoiding re-injury).

In some embodiments, the one or more additional therapies comprise operative intervention, for example, where the subject presents with unrelenting pain, severe impairment of function, or incontinence (which can indicate spinal cord irritation). In some embodiments, the operative intervention comprises removal of the herniated disc with laminotomy (producing a small hole in the bone of the spine surrounding the spinal cord), laminectomy (removal of the bony wall adjacent to the nerve tissues), by needle technique through the skin (percutaneous discectomy), disc-dissolving procedures (chemonucleolysis), and others.

### Musculoskeletal Disease

Musculoskeletal diseases include a multitude of disorders that are prevalent in aging populations (Picavet and Hazes, Ann. Rheum. Dis. 62(7):644-650 (2003), Leveille, Curr. Opin. Rheumatol. 16(2):114-118 (2004), Harkness et al, Rheumatology (Oxford) 44(7):890-895 (2005)). Global population studies and World Health Organization statistics indicate that 10-50% of individuals suffer from musculoskeletal disorders and up to 3% will be classified as disabled due to their bone and joint conditions (Kean and Buchanan, Inflammopharmacology 13(4):343-370 (2005)). The most widely occurring forms of musculoskeletal disease are osteoporosis and osteoarthritis (OA). Globally, OA is estimated to affect 9.6% of men and 18% of women ≥60 years old (Woolf and Pfleger, Bull. World Health Organ. 81(9):646-656 (2003)). Osteoporosis affects as much as 30% of the post-menopausal women in the US, and 23% of women aged ≥50 in the UK (Woolf and Pfleger, Bull. World Health Organ. 81(9):646-656 (2003)).

Musculoskeletal disorder also include: sprains, strains, tendinitis, tenosynovitis, fibromyalgia, osteoarthritis, rheumatoid arthritis, gout, pseudogout (calcium pyrophosphate deposition disease), polymyalgia rheumatica, bursitis, acute and chronic back pain and osteoporosis, which interfere with the normal performance of activities of daily living. Injuries include sprains, strains and tears of ligaments, tendons, muscles and cartilage damage. Pain is the most common symptom and is frequently caused by injury or inflammation. Besides pain, other symptoms such as stiffness, tenderness, weakness and swelling or deformity of affected parts are manifestations of musculoskeletal disorders. Sports injuries are a significant cause of musculoskeletal disorders resulting in pain, strain, sprains, stiffness and leg cramps.

Occupational injuries have become this country's most costly form of illnesses. These disorders account for more than 34% of all workdays lost to injuries and illnesses and cost employers $15 to $20 billion per year in direct workers' compensation costs and another $100 billion on lost productivity, employee turnover, and other indirect expenses. Cumulative trauma disorders (CTDs) frequently involve the upper extremities, such as wrists, shoulders or elbows. Carpal tunnel syndrome of the wrist has become the fastest growing occupational hazard in the United States today.

Musculoskeletal diseases and disorders, particularly age-related musculoskeletal diseases and disorders can be treated with the disclosed compositions and formulations. The compositions described herein can be administered in effective dosages, alone or in combination with a second therapeutic, to improve at least one symptom or manifestation of the disease. As used herein, “improve” means to inhibit the rate of development, stop development, or reverse development of a symptom or characteristic of the disorder. The compositions and formulations can be used to treat, alleviate, reduce, or inhibit a musculoskeletal disease and disorder, particular an age-related musculoskeletal diseases and disorders, senescence, or symptoms thereof.

Exemplary musculoskeletal diseases and disorders include but are not limited to low back pain, pain in the pain in the shoulder, elbow, hip, neck, or foot pain, osteoarthritis, osteoporosis, tendinitis, bursitis, gout, fibromyalgia, rheumatoid arthritis, degenerative changes to muscles, tendons, ligaments, and/or joints, reduced strength and sarcopenia. In some embodiments, the disease or disorder causes pain in muscular or tendinous areas of the extremities but not in joints such as, for example, soft tissue rheumatism. A preferred disease to be treated includes but is not limited to osteoporosis, in particular, age-related osteoporosis.

In some embodiments, the subject is at least 30, 35, 40, 45, 50, 55, 60, 65, 70, 75, 80, 85, 90, or 95 years in age. The subject can be in an age range, for example, the subject can be between 30 and 90 years old, between 35 and 85 years old, between 40 and 80 years old, between 45 and 75 years old, between 50 and 70 years old, or between 55 and 65 years old. In some embodiments the subject is 45-65 years old, 60-65 years old, or 50-70 years old.

In addition, the compositions and formulations disclosed herein can be used for prophylactic treatment of a musculoskeletal disease or disorder, particular an age-related musculoskeletal disease or disorder. Under these circumstances, it may be beneficial to treat these individuals with therapeutically effective doses of the compositions or formulations to reduce the risk of developing a musculoskeletal disease or disorder, particular an age-related musculoskeletal disease or disorder. In other embodiments, the compositions and formulations in a suitable formulation are administered to a subject who has reached a particular age, for example age 40 or more. In yet other embodiments, the compositions and formulations in a suitable formulation are administered to subjects who exhibit symptoms of a musculoskeletal disease or disorder, particular an age-related musculoskeletal disease or disorder. In still other embodiments, the compositions and formulations may be administered to a subject as a preventive measure. In some embodiments, the compositions and formulations in a suitable formulation may be administered to a subject based on demographics or epidemiological studies, or to a subject in a particular field or career.

### Second Therapeutic Compositions and Second Therapies

In any of the above methods of treatment, the method can comprise the administration of a second therapeutic composition or second therapy. The recitation of specific second therapeutic compounds or second therapies in the methods of treating specific diseases, above, are not intended to be exclusive.

In certain embodiments, the second therapeutic compound or second therapy results in a therapeutic synergy with the composition or formulation as disclosed herein. Therapeutic synergy represents a therapeutic effect achieved with a tolerated regimen of a combination treatment that exceeds the optimal effect achieved at any tolerated dose of monotherapy associated with the same therapeutic agents or treatments used in the combination.

In some embodiments, the second therapy comprises application of laser energy to the tissue or part of the body where the disclosed composition or formulation has been administered. Without wishing to be bound to any theory, laser energy can stimulate neurons and may be particularly helpful in the treatment of neuropathy or other indications where nerve damage is present. Laser energy can be applied to the patient before, after, or during the application of the composition or formulation, or any combination thereof. The timing of the application of laser energy relative to the application of the disclosed composition or formulation will depend on a number of factors, including the type of injury, the location of the injury, and pragmatic considerations such as the number of practitioners present or the amount of operating space. That is, laser energy can be applied before, after or during the application of the disclosed composition or formulation, or any combination of timing. In one embodiment, the laser energy is applied at least after the disclosed composition or formulation is applied, and more preferably promptly after the disclosed composition or formulation is applied. Additional laser energy may be applied over the entire extremity containing the injury or disease, any non-injured adjacent tissue, as well as entire body application for stimulation of other body systems such as the lymphatic, circulatory, and nervous systems.

Low-level laser energy serves as a potent agonist to activate the platelets, thereby enhancing the efficacy of the disclosed composition or formulation and eliminating unwanted effects of chemical activators such as thrombin or calcium.

There are a number of variables in determining sufficient and appropriate laser therapy including the wavelength of the laser beam, the area impinged by the laser beam, laser energy, pulse frequency, treatment duration, depth and type of the injury or disease, and tissue characteristics. The wavelength of the applied laser energy depends on the nature of the injury, among other factors, and ranges from ultraviolet to infrared. Preferably, however, the applied laser energy is in the visible spectrum, from about 396 nm to about 800 nm. Pulse frequencies from 0 to 100,000 Hz may be employed to achieve the desired effect on the patient's tissue. When there are no pulses, a continuous beam of laser light is generated. The patient feels no sensation of the low-level laser energy being applied.

Low-level lasers, such as those described in U.S. Pat. Nos. 6,013,096, 6,746,473, and 7,118,588 which are incorporated herein by reference, can be used for treating injuries and diseases with the present method. Hand-held lasers are particularly convenient for treating areas at or near the injury or diseased tissue in a patient's body.

In another embodiment, a hand-held laser device is used to apply laser energy to the patient. The laser device has at least one energy source, preferably a semiconductor laser diode that produces light in the red range of the visible spectrum, having a wavelength of about 635 nm. The laser device includes a rod lens through which the laser light is emitted, creating a line of light. The line of light is scanned across the area of injury or disease.

Alternatively, as explained in more detail in U.S. Pat. No. 7,118,588, the laser device includes a carriage that rotates about an axis that is substantially co-axial to the incident laser beam, thereby causing the laser energy passing through the optical element to sweep through a 360° circle, resulting in a large circular beam spot. The carriage is rotated with a drive assembly. The drive assembly is preferably a main drive gear which is mated with a minor drive gear. The minor drive gear is driven by a main drive motor. The carriage rotates around the axis as the main drive gear is turned. Thus, the laser beam from laser energy source passes through a hollow spindle and strikes an optical element which deflects the laser beam into a linear beam spot that, in combination with the rotation, appears as a circular beam spot. Preferably, the laser beam remains coaxial with the hollow spindle through the optical element, so that the center of the beam spot created by the optical element is on the axis of the hollow spindle.

In some embodiments, the laser energy applied has a wavelength between about 400 nm to 1500 nm, in a constant wave, pulsed, or a combination of both. Pulse frequencies from 0 to 100,000 Hz may be employed. The output power of the laser used to apply the laser energy is between about 1 mW to 500 mW. The laser energy is applied long enough to the disclosed composition or formulation to stimulate the platelets, which usually takes between about 1 second and about 24 hours. Laser stimulation of the disclosed composition or formulation can occur prior to application to the injured or diseased tissue and/or in situ after the composition or formulation has been already been applied to the tissue.

In some embodiments, the compositions and formulations provided herein are administered sequentially or simultaneously with stem cells. Various stem cells known in the art may be used according to the disease or indication to be treated. In certain aspects, amniotic stem cells are administered. Amniotic stem cells are the mixture of stem cells that can be obtained from the amniotic fluid as well as the amniotic membrane. They can develop into various tissue types including skin, cartilage, cardiac tissue, nerves, muscle, and bone. In other aspects, the stem cells are adipose tissue-derived stem cells.

In other aspects, administration of the compositions and formulations provided herein together with hyaluronic acid is advantageous. Hyaluronic acid is a main component of the extracellular matrix, and has a key role in tissue regeneration, inflammation response, and angiogenesis, which are phases of skin wound repair.

### Methods of Administration

The compositions and formulations provided herein may be transplanted into a subject in any pharmaceutically or medically acceptable manner, including by surgical implantation or injection, e.g., intravenous injection, intra-arterial injection, intra-articular injection, intramuscular injection, intraperitoneal injection, intraocular injection, direct injection into a particular tissue.

The site of delivery of the composition is typically at or near the site of pathogenesis, e.g., tissue damage. The site of tissue damage can be determined by well-established methods including medical imaging, patient feedback, or a combination thereof. The particular imaging method used may be determined based on the tissue type. Commonly used imaging methods include, but are not limited to MRI, X-ray, CT scan, Positron Emission tomography (PET), Single Photon Emission Computed Tomography (SPECT), Electrical Impedance Tomography (EIT), Electrical Source Imaging (ESI), Magnetic Source Imaging (MSI), laser optical imaging and ultrasound techniques.

The patient may also assist in locating the site of tissue injury or damage by pointing out areas of particular pain and/or discomfort. The composition or formulation as disclosed herein may be delivered minimally invasively and/or surgically. For example, to deliver the composition or formulation to ischemic tissue, a physician may use one of a variety of access techniques, including but not limited to, surgical (e.g., sternotomy, thoracotomy, mini-thoracotomy, sub-xiphoidal) approaches, endoscopic approaches (e.g., intercostal and transxiphoidal) and percutaneous (e.g., transvascular) approaches.

The composition or formulation may comprise, or be suspended in, any pharmaceutically-acceptable carrier. The combination composition may be carried, stored, or transported in any pharmaceutically or medically acceptable container, for example, a blood bag, transfer bag, plastic tube or vial.

In certain embodiments, the subject is an animal, preferably a mammal, more preferably a non-human primate. In certain embodiments, the subject is a human patient. The subject can be a male or female subject. In certain embodiments, the subject is a non-human animal, such as, for instance, a cow, sheep, goat, horse, dog, cat, rabbit, rat or mouse.

### Kits

Kits may include any device, component, or combination of devices and/or components described herein. For example, the kits may include one or more preparation devices, one or more delivery devices, one or more collection devices, and/or instructions for use. The one or more preparation devices may be for preparing the disclosed compositions and formulations and may comprise a composition or formulation as provided herein to damaged or diseased tissue.

The one or more collection devices may comprise one or more syringes, apheresis needles, or other devices for collecting blood from a patient. The patient may be presently suffering or have suffered one or more of the diseases or indications disclosed herein. The components of the kit may be packaged in sterile containers. The kits may comprise one or more single-use components. Instructions may be in written or pictograph form, or may be on recorded media including audio tape, audio CD, video tape, DVD, CD-ROM, or the like.

Platelet-rich plasma (PRP) has traditionally been identified as having a concentration of 2.5×-5.0× of platelets. The predominant school of thought has been that unless one centrifuges the plasma twice, once at a high rate of speed, and then again, at a slower rate of speed, the resulting product could be properly identified as PRP.

In an embodiment of the present invention, a single centrifugation step is performed. In that single spin a small amount of PRP is produced along with a larger amount of platelet-poor plasma (PPP). PPP is a concentration of less than 2.5× platelets compared to the starting material. For blood banking purposes the most common anticoagulant used is ACD(A) (anhydrous citric acid, dextrose monohydrate, and trisodium citrate dihydrate solution). It has been found to be healthier and less harsh with minimal interference with platelet function. Centrifuge speed is another important factor. Fast spins (tend to damage platelets at a higher level than slow spins. In one embodiment, a centrifugation speed of 3300 rpm produces viable, healthy platelets.

Prior to PRP injections becoming common place, the inventor worked on a project to develop a fibrin clot which could be sutured into a surgical site. This technology would be sold primarily to orthopedic surgeons. A fibrin clot is the highest concentration of platelets possible. Once implanted it would slowly release platelets, bringing down the level of post-operative pain and speeding the healing process. The inventor asked, “What if we stopped short of the clot? What would we have?” The answer is platelets suspended in plasma. At one level it could be considered rich, but the bulk would be poor. There would also be protein, hormones, electrolytes and fibrinogen. Everything needed to heal a wound and to jumpstart tissue rejuvenation and regeneration. This idea provided the foundation for the development of the compositions and formulations disclosed herein.

Without wishing to be bound to any theory, during administration of the disclosed compositions and formulations, as soon as the needle breaks the skin native platelets move into the area because the body recognizes a new injury. Then, the injected activated platelets (i.e., platelets with an optimal amount of calcium chloride and zinc sulfate) rapidly take action to begin the healing and regenerating process. Even at a low concentrations, activated platelets release growth factors. These growth factors signal and recruit native blood borne stem cells as well as dormant stem cells embedded in tissue. The zinc sulfate in the activation solution prompts platelet aggregation to start. Soon after injection, the healing has started at a high rate.

The activation solution has been developed to optimize efficiency while avoiding adverse side effects. Previously, thrombin was identified as the effective way to activate platelets, but due to the potential for allergic reactions in some patients it is not favored. In some embodiments, the compositions or formulations comprise calcium chloride, potassium chloride, calcium gluconate, or a combination thereof. In one embodiment, the compositions or formulations comprise calcium chloride. In the clinical setting, calcium chloride performed well as it relates to reduction of pain on injection and immediate reduction of pain from the injury or condition. In other embodiments, the compositions or formulations comprise zinc sulfate, which promotes tissue healing.

In one aspect of the invention, a small amount of blood is withdrawn from the patient (e.g., about 8.5 to 17 cc). The blood is centrifuged in a basic lab centrifuge with a standard centrifuge protocol of 10 minutes at 3300 rpm. While this is being done, the activation solution is drawn into a 10 cc syringe. The single spin produces both PRP and PPP. It is drawn into the pre-loaded syringe. Platelets are activated instantly, thereby releasing growth factors. Growth factors signal native blood borne stem cells to activate and move to the site of injury. Additionally, tissue embedded stem cells are recruited. The healing process begins instantly. At the same time the zinc sulfate is inducing native platelet aggregation, and another vital step in healing is jumpstarted.

The present invention is further illustrated by the following examples that should not be construed as limiting. The contents of all references, patents, and published patent applications cited throughout this application, as well as the Figures, are incorporated herein by reference in their entirety for all purposes.

### EXAMPLES

### Example 1. Preparation of Platelet Formulations for Musculoskeletal and Neuropathy Procedures

PlasmaPro (“PLP”) is an injectable biologic graft. It contains a high concentration of growth factors which will activate native adult stem cells and provide scaffolding for healing of tendon, ligament, bone and soft tissue injuries and conditions. Additionally, this graft contains high levels of proteins, hormones and fibrinogen, which all work to facilitate healing. When using this treatment in conjunction with stem cell treatments, it is recommended that PLP be used one week prior to the introduction of stem cell grafts. PLP can also be used one week after stem cell treatment. It is not advised to use the two treatments mixed or simultaneously. PLP is alternatively known as “PlasmaPro” or “PlasmaNeu”.

### Technical Process:

For musculoskeletal procedures including acute injuries and chronic conditions:
a. Draw 8.5 cc to 17.0 cc of the patient's whole blood into yellow-top acid citrate dextrose (ACD) tube(s).
b. Place the tube(s) in a centrifuge and set the cycle for 10 minutes at 3300 rpm.
c. At the end of the spin, extract the plasma from one or both tubes into a syringe preloaded with a PLP Activation/Aggregation Solution (see Table 1).
   For neuropathy procedures:
a. Draw 17.0 cc of the patient's blood into two yellow-top ACD tubes.
b. Place the tubes in a centrifuge and set the cycle for 10 minutes at 3300 rpm.
c. At the conclusion of the spin, extract the plasma from the tubes into a syringe pre-loaded with a PLP Activation/Aggregation Solution (see Table 1).

### Treatment:

PLP should be injected directly into the point of injury according to a treatment protocol (see Example 5).

### Example 2. Schedule for Treatment Protocols

The protocols were developed to give the maximum benefit to the patient in the least amount of time.

### 1. Injections:

a. Three injection sites, 1 time per week for 6 weeks, optionally performed under diagnostic ultrasound guidance.
   1. Re-evaluate in four weeks; if the patient is 30-50% improved then continue for the remainder of the 6 week treatment.
   2. After completing 6 weeks of the plasma treatment, re-evaluate in 4 weeks. If the patient reports less than a 70% improvement, then monitor for 4-6 months as studies have shown regeneration continues 6 months after the last plasma treatment.
   3. If mild symptoms remain after 6 months then the patient can receive a booster treatment of 1 injection per week for an additional 2 weeks. Re-evaluate in 4 weeks.
   4. If there is no improvement despite 4 weeks of plasma treatment, re-evaluate the patient's compliance with the recommended diet, supplements and their overall health (i.e. the etiology of the peripheral neuropathy).
      Note: Treatment is warranted when symptoms are still present, whether the symptom is even mild. The success of the peripheral neuropathy program is to enhance the function of the damaged/dying nerve to its maximal level. Stopping treatment too soon may allow symptoms to return unnecessarily.
      Additional therapeutic regimens that may accompany the injection of plasma treatment include:
Laser—4 to 5 minutes, 3 times a week, per site.
Vibration—Peripheral neuropathy diagnosis, radicular diagnosis, lumbar/sacrum segmental dysfunction, DDD lumbar.
LSO/DAKOTA® (back and abdomen supporters)—Radiculopathy, Double Crush, DDD cervical/lumbar, spondylolisthesis.
TENS and TENS Garments—Pain relief sock, glove, lumbar or knee wrap. TENS units use either low frequencies (2-4 Hz) or high frequencies (80-100 Hz) to block pain impulses to the brain. This aids the body in producing natural substances that relieve pain and increases blood flow to the injured area.
Healthy Diet—50-60% of the success with neuropathy can be contributed to tracking the patient's food intake to help him or her make better decisions on what they eat. Weekly tracking along with Sugar Tracking is KEY to the patient beating neuropathy of any kind.
Dietary Supplements:
   Tuna Omega 3—EPA and DHA for immune/circulatory health
   NEUROPLEX™ (gabapentin)—Maintains nerve cell health
   Ribonucleic Acid (RNA)—Stop nerve degeneration by improving T-cell and B-cell activity
   CATAPLEX® B (thiamin, niacin, Vitamin B₆): helps maintain healthy nerves

### Nerve Injection Procedure—Lower Extremity

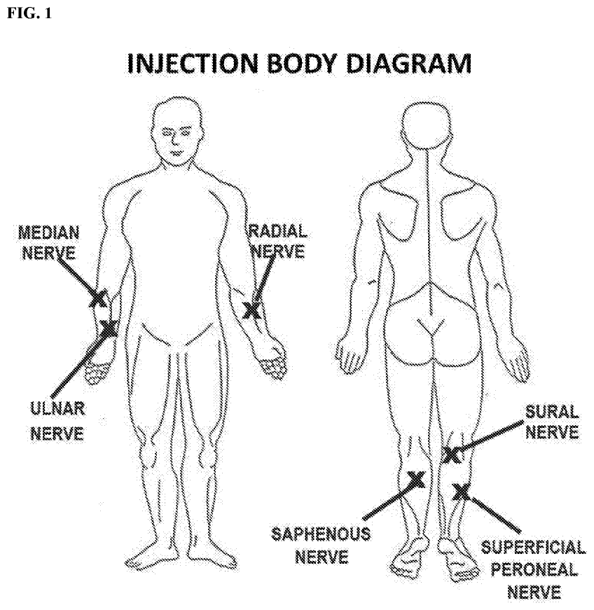

1. Patient is placed on the table in prone position with feet hanging over the end of the table.
2. Mark each nerve according to its anatomical location (see FIG. 1):
   a. Sural nerve—slightly lateral to the midline of the calf
   b. Superficial peroneal nerve—lateral aspect of calf
   c. Saphenous nerve—medial aspect of calf
   Note: Inject 3 nerves on one extremity and then 2 nerves on the other extremity. Alternate the nerve injections each week so both extremities receive sufficient plasma.
3. Utilize the diagnostic ultrasound to confirm the nerve's location and to adjust your mark if necessary.
4. Cleanse each site with a BETADINE® (povidone-iodine) swab and allow to dry. Have prepared a 10 cc syringe of PLP in a 25 gauge 1.5 inch needle.
5. Insert the needle halfway at a 45 degree angle laterally. Pull back on plunger; if no blood then return, and inject 1 cc.
   a. Pull needle almost completely out and reposition the needle at another 45 degree angle medially and inject another 1 cc.
   b. Each nerve has a total of 2 cc injected.
   c. Continue same pattern for each nerve.
6. After injections are completed, apply a 4×4 gauze sponge to each site and then apply bandages.
7. Optionally, utilize laser therapy immediately following the injections to each site.

### Nerve Injection Procedure—Upper Extremity

1. Patient is placed on the table in sitting position with arms to the rotated palm side up, then mark each nerve according to its anatomical location below anteculial fossa (see FIG. 1):
   a. Radial nerve—lateral aspect of arm
   b. Median nerve—slightly medial to midline of arm
   c. Ulnar nerve—medial aspect of arm
   Note: 2 nerves on one extremity will be treated and then 2 nerve on the other extremity to be treated
2. Utilize the diagnostic ultrasound to confirm the nerves location and to adjust your mark if necessary.
3. Cleanse each site with a BETADINE® (povidone-iodine) swab and allow to dry. Have prepared 10 cc syringe of PLP in a 25 gauge 1.5 inch needle.
4. Insert the needle halfway at a 45 degree angle laterally. Pull back on plunger, if no blood return, and inject 1 cc.
   a. Pull needle almost completely out and reposition the needle at another 45 degree angle medially and inject another 1 cc.
   b. Each nerve has a total of 2 cc injected.
   c. Continue same pattern for each nerve.
5. After injections are completed, apply a 4×4 gauze sponge to each site and then apply bandages.
6. Optionally, utilize the laser therapy immediately following the injections to each site.

### Contraindications for the Peripheral Neuropathy Treatment Include:

1. Renal failure
2. Liver failure
3. Cancer—active
4. Chemo—radiation—active
5. Metal implants—in the extremities
6. Peripheral vascular disease
7. Stent placement in the extremity
8. Pitting edema in the extremity
9. Absent or decreased pulse in the extremity
10. Immune system compromised
11. Infection—localized in the extremity or systemic
12. Skin breakdown
13. Febrile >99 degrees
14. Deep Vein Thrombosis
15. Uncontrolled hypertension
16. Chronic symptomatic anemia

### Example 4. Materials List from a Platelet Formulation Kit

The following list of materials is a non-limiting example of a kit that can be used to administer the compositions and formulations provided herein:
1. Two gauze sponges
2. PlasmaGenix Peripheral Neuropathy (Plasma Neu activation solution); the activation solution will contain an optimal amount of calcium chloride and zinc sulfate as disclosed herein to achieve the desired result based on the disease or indication (see Table 1).
3. Three Band-Aids per extremity
4. Two 25 gauge needle, 1.5 inches long and syringe
5. One Butterfly venipuncture needle
6. Two ACD venipuncture tubes
7. One extraction 22 gauge spinal needle

### Additional Material:

1. MEDLIGHT™ 630 PRO Laser—for post-injection treatment
2. Vibrational platform—for balance and coordination therapy, stabilization of joint segments

### Example 5. Treatment Protocols for Musculoskeletal Disorders

Note: Unless noted, all treatments are processed for 10 minutes, with a centrifuge speed of about 3300 rpm.

### Shoulder Disorder

1. Tendon
   a. PlasmaPro: Inject 2-4 ml per tendon for Acute injuries
   b. PlasmaPro: Inject 2-4 ml per tendon for Sub-acute injuries.
   c. PlasmaPro: Inject 2-4 ml per tendon for Chronic injuries
   d. 3-4 injection procedures in 2 week intervals depending upon patients progression
2. Ligament
   a. PlasmaPro: Inject 2-4 ml per tendon for Acute injuries
   b. PlasmaPro: Inject 2-4 ml per tendon for Sub-acute injuries.
   c. PlasmaPro: Inject 2-4 ml per tendon for Chronic injuries
   d. 3-4 injection procedures in 1 OR 2 week intervals depending upon patient's progression.
3. Joint conditions
   a. PlasmaPro: Inject (intra-articular) 5-6 ml for Acute injuries.
   b. PlasmaPro: Inject (intra-articular) 5-6 ml for Sub-acute injuries.
   c. PlasmaPro: Inject (intra-articular) 5-6 ml for Chronic injuries.
   d. 3-4 injection procedures in 1 OR2 week intervals depending upon patients progression

### Elbow Disorder

1. Tendon
   a. PlasmaPro: Inject 2-3 ml per tendon for Acute injuries
   b. PlasmaPro: Inject 2-3 ml per tendon for Sub-acute injuries.
   c. PlasmaPro: Inject 2-3 ml per tendon for Chronic injuries
   d. 3-4 injection procedures in 2 week intervals depending upon patients progression
2. Ligament
   a. PlasmaPro: Inject 2-3 ml per tendon for Acute injuries
   b. PlasmaPro: Inject 2-3 ml per tendon for Sub-acute injuries.
   c. PlasmaPro: Inject 2-3 ml per tendon for Chronic injuries
   d. 3-4 injection procedures in 2 week intervals depending upon patients progression
3. Joint conditions
   a. PlasmaPro: Inject (intra-articular) 3-4 ml for Acute injuries.
   b. PlasmaPro: Inject (intra-articular) 3-4 ml for Sub-acute injuries.
   c. PlasmaPro: Inject (intra-articular) 3-4 ml for Chronic injuries.
   d. 3-4 injection procedures in 2 week intervals depending upon patients progression

### Wrist Disorder

1. Tendon
   a. PlasmaPro: Inject 2-3 ml per tendon for Acute injuries
   b. PlasmaPro: Inject 2-3 ml per tendon for Sub-acute injuries.
   c. PlasmaPro: Inject 2-3 ml per tendon for Chronic injuries
   d. 3-4 injection procedures in 1 OR 2 week intervals depending upon patients progression
2. Ligament
   a. PlasmaPro: Inject 2-3 ml per tendon for Acute injuries
   b. PlasmaPro: Inject 2-3 ml per tendon for Sub-acute injuries.
   c. PlasmaPro: Inject 2-3 ml per tendon for Chronic injuries
   d. 3-4 injection procedures in 2 week intervals depending upon patients progression
3. Joint conditions
   a. PlasmaPro: Inject (intra-articular) 3 ml for Acute injuries.
   b. PlasmaPro: Inject (intra-articular) 3 ml for Sub-acute injuries.
   c. PlasmaPro: Inject (intra-articular) 3 ml for Chronic injuries.
   d. 3-4 injection procedures in 2 week intervals depending upon patients progression

### Hand Disorder

1. Tendon
   a. PlasmaPro: Inject 2-3 ml per tendon for Acute injuries
   b. PlasmaPro: Inject 2-3 ml per tendon for Sub-acute injuries.
   c. PlasmaPro: Inject 2-3 ml per tendon for Chronic injuries
   d. 3-4 injection procedures in 1 OR 2 week intervals depending upon patients progression
2. Ligament
   a. PlasmaPro: Inject 2-3 ml per ligament for Acute injuries
   b. PlasmaPro: Inject 2 ml per ligament for Sub-acute injuries.
   c. PlasmaPro: Inject 3 ml per tendon for Chronic injuries
   d. 3-4 injection procedures in 2 week intervals depending upon patients progression
3. Joint conditions
   a. PlasmaPro: Inject (intra-articular) 2 ml for Acute injuries.
   b. PlasmaPro: Inject (intra-articular) 2 ml for Sub-acute injuries.
   c. PlasmaPro: Inject (intra-articular) 3 ml for Chronic injuries.
   d. 3-4 injection procedures in 2 week intervals depending upon patients progression

### Ankle Condition

1. Tendon
   a. PlasmaPro: Inject 2-4 ml per tendon for Acute injuries
   b. PlasmaPro: Inject 2-4 ml per tendon for Sub-acute injuries.
   c. PlasmaPro: Inject 2-4 ml per tendon for Chronic injuries
   d. 3-4 injection procedures in 2 week intervals depending upon patients progression
2. Ligament
   a. PlasmaPro: Inject 2-4 ml per tendon for Acute injuries
   b. PlasmaPro: Inject 2-4 ml per tendon for Sub-acute injuries.
   c. PlasmaPro: Inject 2-4 ml per tendon for Chronic injuries
   d. 3-4 injection procedures in 2 week intervals depending upon patients progression
3. Joint conditions
   a. PlasmaPro: Inject (intra-articular) 5-6 ml for Acute injuries.
   b. PlasmaPro: Inject (intra-articular) 5-6 ml for Sub-acute injuries.
   c. PlasmaPro: Inject (intra-articular) 5-6 ml for Chronic injuries.
   d. 3-4 injection procedures in 1 OR 2 week intervals depending upon patients progression

### Hip Disorder

1. Tendon
   a. PlasmaPro: Inject 4-5 ml per tendon for Acute injuries
   b. PlasmaPro: Inject 4-6 ml per tendon for Sub-acute injuries.
   c. PlasmaPro: Inject 4-6 ml per tendon for Chronic injuries
   d. 3-4 injection procedures in 2 week intervals depending upon patients progression
2. Ligament
   a. PlasmaPro: Inject 4-5 ml per tendon for Acute injuries
   b. PlasmaPro: Inject 4-5 ml per tendon for Sub-acute injuries.
   c. PlasmaPro: Inject 4-5 ml per tendon for Chronic injuries
   d. 3-4 injection procedures in 2 week intervals depending upon patients progression
3. Joint conditions
   a. PlasmaPro: Inject (intra-articular) 5-6 ml for Acute injuries.
   b. PlasmaPro: Inject (intra-articular) 5-6 ml for Sub-acute injuries.
   c. PlasmaPro: Inject (intra-articular) 5-6 ml for Chronic injuries.
   d. 3-4 injection procedures in 2 week intervals depending upon patients progression

### Knee Disorder

1. Tendon
   a. PlasmaPro: Inject 4-5 ml per tendon for Acute injuries
   b. PlasmaPro: Inject 4-5 ml per tendon for Sub-acute injuries.
   c. PlasmaPro: Inject 4-5 ml per tendon for Chronic injuries
   d. 3-4 injection procedures in 1 OR 2 week intervals depending upon patients progression
2. Ligament
   a. PlasmaPro: Inject 4-5 ml per tendon for Acute injuries
   b. PlasmaPro: Inject 4-5 ml per tendon for Sub-acute injuries.
   c. PlasmaPro: Inject 4-5 ml per tendon for Chronic injuries
   d. 3-4 injection procedures in 1 OR 2 week intervals depending upon patients progression
3. Joint conditions
   a. PlasmaPro: Inject (intra-articular) 5-6 ml for Acute injuries.
   b. PlasmaPro: Inject (intra-articular) 5-6 ml for Sub-acute injuries.
   c. PlasmaPro: Inject (intra-articular) 5-6 ml for Chronic injuries.
   d. 3-4 injection procedures in 1 OR 2 week intervals depending upon patients progression

### Neck Disorder

1. Cervical ligament (Interspinous ligaments)
   a. PlasmaPro: Inject 2 ml per ligament for Acute injuries
   b. PlasmaPro: Inject 2 ml per ligament for Sub-acute injuries
   c. PlasmaPro: Inject 2 ml per ligament for Chronic injuries
   d. 3-4 injection procedures in 2 week intervals depending upon patients progression
2. Cervical paravertebral muscles
   a. PlasmaPro: Inject 2 ml per ligament for Acute injuries
   b. PlasmaPro: Inject 2 ml per ligament for Sub-acute injuries
   c. PlasmaPro: Inject 2 ml per ligament for Chronic injuries
   d. 3-4 injection procedures in 2 week intervals depending upon patients progression
3. Cervical facet joints
   a. PlasmaNeu: Inject 2 ml per facet joint for Acute injuries.
   b. PlasmaNeu: Inject 2 ml per facet joint for Sub-acute injuries.
   c. PlasmaPro: Inject 2 ml per facet joint for Chronic injuries.
   d. 3-4 injection procedures in 2 week intervals depending upon patients progression and chronicity of condition

### Mid-Back Disorder

1. Thoracic ligament (Interspinous ligaments)
   a. PlasmaPro: Inject 2 ml per ligament for Acute injuries
   b. PlasmaPro: Inject 2 ml per ligament for Sub-acute injuries
   c. PlasmaPro: Inject 2 ml per ligament for Chronic injuries
   d. 3-4 injection procedures in 2 week intervals depending upon patients progression
2. Thoracic paravertebral muscles
   a. PlasmaPro: Inject 2 ml per ligament for Acute injuries
   b. PlasmaPro: Inject 2 ml per ligament for Sub-acute injuries
   c. PlasmaPro: Inject 2 ml per ligament for Chronic injuries
   d. 3-4 injection procedures in 2 week intervals depending upon patients progression
3. Thoracic facet joints
   a. PlasmaPro: Inject 2 ml per facet joint for Acute injuries.
   b. PlasmaPro: Inject 2 ml per facet joint for Sub-acute injuries.
   c. PlasmaPro: Inject 2 ml per facet joint for Chronic injuries.
   d. 3-4 injection procedures in 2 week intervals depending upon patients progression and chronicity of condition

### Lower Back Disorder

1. Lumbosacral ligaments
   a. PlasmaPro: Inject 2 ml per ligament for Acute injuries
   b. PlasmaPro: Inject 2 ml per ligament for Sub-acute injuries
   c. PlasmaPro: Inject 2 ml per ligament for Chronic injuries
   d. 3-4 injection procedures in 2 week intervals depending upon patients progression
2. Lumbosacral paravertebral muscles
   a. PlasmaPro: Inject 2 ml per ligament for Acute injuries
   b. PlasmaPro: Inject 2 ml per ligament for Sub-acute injuries
   c. PlasmaPro: Inject 2 ml per ligament for Chronic injuries
   d. 3-4 injection procedures in 2 week intervals depending upon patients progression
3. Lumbar facet joints
   a. PlasmaPro: Inject 2 ml per facet joint for Acute injuries.
   b. PlasmaPro: Inject 2 ml per facet joint for Sub-acute injuries.
   c. PlasmaPro: Inject 2 ml per facet joint for Chronic injuries.
   d. 3-4 injection procedures in 2 week intervals depending upon patients progression and chronicity of condition
4. Sciatica
   a. PlasmaPro: Inject 4-5 ml directly into the musculature of the gluteus maximus.
b. 3-4 injection procedures in 1 week intervals depending upon patient's progress.

### Example 6. Treatment Protocol for Wound Healing

The following protocol has been designed for treating skin wounds. The steps of producing and administering a clot containing platelets are optional. However, the combined effect of the clot in the wound bed and the administration of the platelets to the periphery of the wound may accelerate healing.
1. Draw 8.5 cc to 17.0 cc of the patient's whole blood into yellow-top acid citrate dextrose (ACD) tube(s) to produce the injectable platelet formulation (i.e., PLP).
2. Draw similar volumes of the patient's whole blood into a tube containing a clot activator.
3. Place the tubes in a centrifuge and set the cycle for 10 minutes at 3300 rpm.
4. At the conclusion of the spin, extract the plasma from the ACD tubes into a syringe pre-loaded with a PLP Activation/Aggregation Solution (see Table 1). Also, carefully remove the platelet clot from the other tubes.
5. Inject the activated platelet formulation at the periphery of the wound and carefully place the clot in the wound bed.
6. Cover the wound with a clean bandage and monitor healing of the wound at each subsequent administration of platelets (see Table 1).

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications, patents, and patent publications cited are incorporated by reference herein in their entirety for all purposes.

The publications discussed herein are provided solely for their disclosure prior to the filing date of the present application. Nothing herein is to be construed as an admission that the present invention is not entitled to antedate such publication by virtue of prior invention.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

What is claimed is:

1. A pharmaceutical composition comprising platelets, calcium chloride, and zinc sulfate, wherein the weight ratio of calcium chloride to zinc sulfate is between about 500:1 and about 10:1.

2. The pharmaceutical composition according to claim 1, wherein the platelets are in a concentration of about 100,000/μl to about 1,000,000/μl.

3. The pharmaceutical composition according to claim 1, wherein the weight ratio of calcium chloride to zinc sulfate is between about 350:1 and about 100:1.

4. The pharmaceutical composition according to claim 1, wherein the combined concentration of calcium chloride and zinc sulfate is less than about 500 mg/mL.

5. The pharmaceutical composition according to claim 4, wherein the combined concentration of calcium chloride and zinc sulfate is between about 25 mg/mL and about 300 mg/mL.

6. The pharmaceutical composition according to claim 1, further comprising cytokines, chemokines, inflammatory mediators, and growth factors.

7. The pharmaceutical composition according to claim 1, wherein the composition is formulated as a liquid, a solid, a semi-solid, or a combination thereof.

8. A method of treating a wound, chronic pain, neuropathy, orthopedic injury, or musculoskeletal disease, the method comprising:
administering to a subject in need thereof an effective amount of a pharmaceutical composition comprising platelets, calcium chloride, and zinc sulfate,
wherein the weight ratio of calcium chloride to zinc sulfate is between about 500:1 and about 10:1, and
administering to the subject a second therapeutic agent or a second therapy.

9. The method according to claim 8, wherein the wound is an epidermal wound, skin wound, chronic wound, acute wound, external wound, internal wound, or congenital wound.

10. The method according to claim 8, wherein the chronic pain or neuropathy is, or is caused by, neuritis, diabetes mellitus, peripheral neuropathy, reflex sympathetic dystrophy syndrome, phantom limb pain, post-amputation pain, postherpetic neuralgia, shingles, central pain syndrome, Guillain-Barre Syndrome, degenerative disc disease, cancer, multiple sclerosis, kidney disorders, alcoholism, human immunodeficiency virus-related neuropathy, Wartenberg's Migratory Sensory Neuropathy, fibromyalgia syndrome, causalgia, spinal cord injury, or exposure to a chemical agent.

11. The method according to claim 8, wherein the orthopedic injury is a bone defect, disc herniation or degenerative disc disease.

12. The method according to claim 8, wherein the musculoskeletal disease is selected from the group consisting of shoulder, elbow, hip, neck, or foot pain, osteoarthritis, osteoporosis, tendinitis, bursitis, gout, fibromyalgia, rheumatoid arthritis, degenerative changes to muscles, tendons, ligaments, or joints, reduced strength, sarcopenia, and soft tissue rheumatism.

13. The method according to claim 8, wherein the second therapeutic agent or second therapy is administered simultaneously with the pharmaceutical composition.

14. The method according to claim 8, wherein the second therapeutic agent or second therapy is administered sequentially with the pharmaceutical composition.

15. The method according to claim 8, wherein the second therapeutic agent or second therapy comprises stem cells.

16. The method according to claim 15, wherein the stem cells are amniotic stem cells or adipose tissue-derived stem cells.

17. The method according to claim 8, wherein the weight ratio of calcium chloride to zinc sulfate is between about 350:1 and about 100:1.

18. The method according to claim 8, wherein the combined concentration of calcium chloride and zinc sulfate is less than about 500 mg/mL.

19. The method according to claim 18, wherein the combined concentration of calcium chloride and zinc sulfate is between about 25 mg/mL and about 300 mg/mL.

20. The method according to claim 8, wherein the pharmaceutical composition further comprises cytokines, chemokines, inflammatory mediators, and growth factors.
